(19) United States
(12) Patent Application Publication
(10) Pub. No.: US 2025/0368970 A1
(43) Pub. Date: Dec. 4, 2025

(54) METHOD FOR PURIFYING FACTOR XIII

(71) Applicant: GREEN CROSS CORPORATION, Yongin-si (KR)

(72) Inventors: Taehyun BAN, Yongin-si (KR); Eun BANG, Yongin-si (KR); Kang Yun SEO, Yongin-si (KR); Il Ha JANG, Yongin-si (KR); Min-Jung KIM, Yongin-si (KR)

(73) Assignee: GREEN CROSS CORPORATION, Yongin-si (KR)

(21) Appl. No.: 18/726,275

(22) PCT Filed: Feb. 10, 2023
(86) PCT No.: PCT/KR2023/002024

§ 371 Date: Jul. 2, 2024

(30) Foreign Application Priority Data
Feb. 11, 2022 (KR) 10-2022-0018141

(51) Int. Cl. C12N 9/10

(52) U.S. Cl. CPC C12N 9/1044; C12Y 203/02013

(57) ABSTRACT

A method of purifying factor XIII (FXIII), including performing a first salt precipitation by adding diatomite and a salt to a separated plasma sample containing factor XIII, and then obtaining a precipitate by removing a supernatant, dissolving the precipitate in a dissolution buffer, and then treating with heat to form a solution, and performing a second salt precipitation by adding a salt to the solution and then obtaining a precipitate by removing a supernatant.

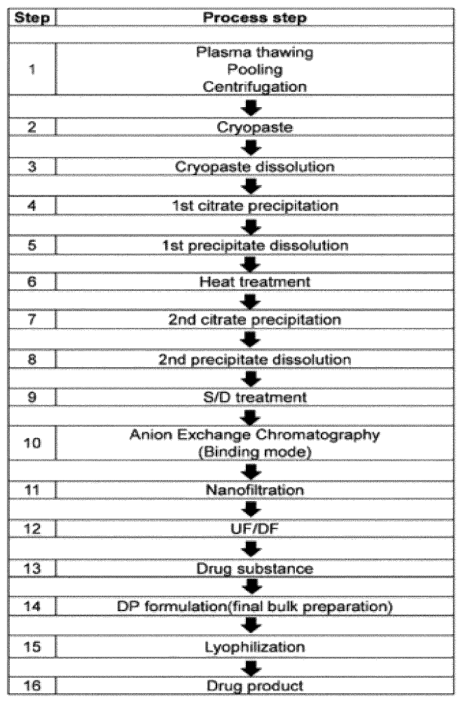

### CROSS REFERENCE TO RELATED APPLICATIONS

This application is a National Stage entry under 35 U.S.C. § 371 of PCT/KR2023/002024, filed on Feb. 10, 2023, and claims priority to Korean Patent Application No. 10-2022-0018141, filed on Feb. 11, 2022. The entire contents of both are incorporated herein by reference.

### TECHNICAL FIELD

The present invention relates to a method of purifying factor XIII, and more particularly to a method of purifying factor XIII, comprising (a) precipitating by adding diatomite and a salt to a separated plasma sample containing factor XIII, and then obtaining a precipitate by removing a supernatant (first salt precipitation); (b) obtaining a solution by dissolving the precipitate obtained by first salt precipitation in step (a) in a dissolution buffer, and then treating heat (heat treatment); and (c) precipitating by adding a salt to the solution heat-treated in step (b), and then obtaining a precipitate by removing a supernatant (second salt precipitation).

### BACKGROUND ART

Factor XIII (FXIII, fibrin stabilizing factor (FSF), Factor 13) is a zymogen complexed with fibrinogen and is a plasma glycoprotein circulating in the blood (Greenberg and Shu an, J. Biol. Chem. 257:6096-6101, 1982). Plasma factor XIII zymogen is a tetramer composed of two a subunits and two b subunits (Chung et al., J. Biol. Chem. 249:940-950, 1974), in which the a subunit includes the catalytic site of the enzyme and the b subunit is known to stabilize the a subunit or modulate the activation of factor XIII (Lorand et al., Biochem. Biophys. Res. Comm. 56:914-922, 1974). The amino acid sequences of the a and b subunits are known (Ichinose et al., Biochemistry 25:6900-6906, 1986).

Factor XIII (factor XIIIa) activated in vivo catalyzes a cross-linking reaction between different protein molecules. In the final stage of blood coagulation, thrombin converts the factor XIII zymogen into an intermediate form (a′2b2), which then dissociates in the presence of calcium ions to produce factor XIIIa, which is a homodimer of the a subunit. Factor XIIIa is a transglutaminase that increases coagulation strength by catalyzing the cross-linking of fibrin polymers (Chen and Doolittle, Proc. Natl. Acad. Sci. USA 66:472-479, 1970).

Factor XIII is useful in postoperative wound healing (Mishi A et al., Chirurcr 55:803-808, 1984) and in the treatment of patients suffering from scleroderma (Grivaux and Pieron, Rev. Pnemol. Clin. 43:102-103, 1987), ulcerative colitis (Suzuki and Takamura, Thromb. Haemostasis. 58:509, 1987), and pseudomembranous colitis (Kuratsuji et al., Haemostasis, 11:229-234, 1982) and the prevention of rebleeding in patients suffering from subarachnoid hemorrhage (Henze et al., Thromb. Haemostas. 58:513, 1987). Factor XIII has also been used as a component of tissue adhesives (U.S. Pat. No. 4,414,976, et al.).

Several methods are known for the purification of factor XIII. The production of factor XIII from platelet-enriched plasma or fibrinogen preparations is known (Chung and Folk, J. Biol. Chem. 247:2798-2807, 1972). Purification of factor XIII from the Cohn-I fraction is known, which includes several ammonium sulfate precipitation steps and DEAE cellulose fractionation (Cooke and Holbrook, Biochem. J. 141:79-84, 197 4). Methods of purifying the a subunit of factor XIII from placental concentrates using chromatography and ammonium sulfate precipitation are known (Skrzynia et al., Blood 60:1089-1095, 1985), and U.S. Pat. No. 4,597,899 discloses the isolation of factor XIII from placental extracts by alcohol precipitation.

Against this technical background, the present inventors have made great efforts to increase the purification yield and purity of factor XIII, and ascertained that, in the factor XIII purification process comprising a precipitation step using a salt such as citrate, etc., there may occur a problem upon application to a production scale later due to a phenomenon by which the precipitate adheres to an impeller, and thus, conditions able to prevent the precipitate from adhering through addition of an auxiliary material in the salt precipitation step are specified, thereby culminating in the present invention.

The information described in this background section is only for improving under-standing of the background of the present invention, and is not to be construed as including information forming the related art already known to those of ordinary skill in the art to which the present invention belongs.

### SUMMARY OF THE INVENTION

It is an object of the present invention to provide a method of purifying factor XIII with high yield and high purity, which is capable of increasing the efficiency of a precipitation step using a salt such as citrate or the like.

In order to accomplish the above object, the present invention provides a method of purifying factor XIII, comprising (a) precipitating by adding diatomite and a salt to a separated plasma sample containing factor XIII, and then obtaining a precipitate by removing a supernatant (first salt precipitation); (b) obtaining a solution by dissolving the precipitate obtained by first salt precipitation in step (a) in a dissolution buffer, and then treating heat (heat treatment); and (c) precipitating by adding a salt to the solution heat-treated in step (b), and then obtaining a precipitate by removing a supernatant (second salt precipitation).

### BRIEF DESCRIPTION OF DRAWINGS

FIGURE schematically shows a process of purifying factor XIII according to the present invention.

### DETAILED DESCRIPTION AND PREFERRED EMBODIMENTS OF THE INVENTION

Unless otherwise defined, all technical and scientific terms used herein have the same meanings as those typically understood by those skilled in the art to which the present invention belongs. Generally, the nomenclature used herein is well known in the art and is typical.

“Process,” “purification,” or “separation,” as used interchangeably herein may indicate the use of one or more methods or devices to achieve a particular result (e.g. purification of factor XIII) in a purification process.

In one aspect, the present invention is directed to a method of purifying factor XIII, comprising:
(a) precipitating by adding diatomite and a salt to a separated plasma sample containing factor XIII, and then obtaining a precipitate by removing a supernatant (first salt precipitation);
(b) obtaining a solution by dissolving the precipitate obtained by first salt precipitation in step (a) in a dissolution buffer, and then treating heat (heat treatment); and
(c) precipitating by adding a salt to the solution heat-treated in step (b), and then obtaining a precipitate by removing a supernatant (second salt precipitation).

In the present invention, the plasma sample may be a cryopaste obtained from human plasma, and is prepared through cold centrifugation.

The cryopaste may be dissolved using a dissolution buffer at a mass ratio of 1:1 to 1:7, preferably 1:2 to 1:5, most preferably 1:2 to 1:4, and a dissolution buffer containing 20 mM sodium citrate and 50 mM NaCl (pH 7.0) may be used, but the present invention is not limited thereto.

In the present invention, the diatomite that is added in step (a) accounts for 0.5 to 5% (w/v), preferably 1 to 3% (w/v) of the total volume, but the present invention is not limited thereto.

In the present invention, “diatomite” is a sedimentary silica mineral composed of the fossilized skeletal remains of single-celled algae-like plants called diatoms that were deposited in sea or freshwater environments. The honeycomb silica structure imparts diatomite with useful properties, such as absorption capacity, surface area, chemical stability, and low bulk density.

In the present invention, the diatomite may comprise 96 to 99% of SiO₂ (silicon dioxide), and may further comprise at least one selected from the group consisting of Al₂O₃, Fe₂O₃, Na₂O, K₂O, MgO, CaO, TiO₂, P₂O₅, and MnO₂, but the present invention is not limited thereto.

In the present invention, the diatomite in step (a) may be Celpure®, particularly Celpure® 1000 or Celpure® 300, but is not limited thereto.

Preferably, the composition of Celpure® 300 is as shown in Table 1 below, and Celpure® 1000 has a composition similar to Celpure® 300 but has a difference in permeability as shown in Table 2 below, but the present invention is not limited thereto.

TABLE 1
Celpure ® 300
(high purity grade)

| | |
|---|---|
| SiO₂ | 98.65% |
| Al₂O₃ | 0.60 |
| Fe₂O₃ | 0.27 |
| Na₂O | 0.14 |
| K₂O | 0.10 |
| MgO | 0.08 |
| CaO | 0.08 |
| TiO₂ | 0.03 |
| P₂O₅ | 0.03 |
| MnO₂ | nd* |
| Total | 99.98 |

*nd Below detectable limit

TABLE 2

| Celpure ® | Permeability | Surface Area | 99% Retention |
|---|---|---|---|
| Grade | (mDarcy) | (m²/g) | (micron*) |
| 300 | 150-300 | 3.0-4.0 | <0.65 |
| 1000 | 750-1,250 | 1.0-2.0 | <2.0 |

*Data is provided for comparison purposes only. Depending on the compressiblity of the solids, values may vary from those in the table.

In an embodiment of the present invention, the purification yield was confirmed to decrease due to a phenomenon by which the precipitate in the first salt precipitation step adheres to an impeller that is a blade used for liquid stirring, and in order to compensate therefor, a diatomite filter aid was added as an auxiliary material.

In the present invention, the salt used in each of steps (a) and (c) may be citrate, ethanol, glycine, ammonium sulfate ((NH4)2SO4), PEG, or barium chloride (BaC12), preferably citrate, more preferably sodium citrate, but is not limited thereto. Any salt may be used without limitation, so long as it is commonly used for salt precipitation.

In the present invention, the concentration of sodium citrate in step (a) may be 250 to 400 mM, preferably 310 to 350 mM, more preferably 320 to 340 mM, and the concentration of sodium citrate in step (c) may be 500 to 800 mM, preferably 640 to 700 mM, more preferably 640 to 660 mM.

In the present invention, in the first and second salt precipitation steps, a dissolution buffer common in the art may be further used to dissolve the precipitate. In an embodiment, ultra-pure water (UPW) was used as the dissolution buffer, but is not limited thereto.

In an embodiment of the present invention, obtaining the precipitate by the first salt precipitation may be performed through addition of a salt, stirring at 200 to 500 rpm at 25 to 31° C., and then centrifugation at 1 to 10° C. and 5000 to 8000 g for 30 minutes, but the present invention is not limited thereto.

In an embodiment of the present invention, obtaining the precipitate by the second salt precipitation may be performed through addition of a salt, stirring at 200 to 500 rpm at 27 to 35° C., and then centrifugation at 1 to 10° C. and 5000 to 8000 g for 30 minutes, but the present invention is not limited thereto.

In the present invention, a temperature of the heat treatment in step (b) may be 40 to 70° C., preferably 50 to 60° C., more preferably 53 to 57° C., but the present invention is not limited thereto.

In the present invention, the heat treatment may be performed by adding AlOH3 (aluminum hydroxide) as an adsorbent, but is not limited thereto. AlOH3 adsorption gel, which is an adsorption precipitate on which impurities are adsorbed, may be removed after centrifugation. AlOH3 may be used at a concentration of 1 to 7% (w/v), preferably 2 to 6% (w/v), more preferably 3 to 5% (w/v), but the present invention is not limited thereto.

In the present invention, the purification method may further comprise, after dissolution of the obtained precipitate after step (c),
(d) treating solvent/detergent (SID treatment); and/or
(e) performing anion exchange chromatography.

In the present invention, any solvent and detergent may be used without limitation, so long as they are able to inactivate a virus, particularly a lipid-enveloped virus. The detergent may be selected from the group consisting of nonionic and ionic detergents, and is preferably substantially a non-denaturing detergent. In particular, for ease of removal, a nonionic detergent is preferable, and the solvent is most preferably tri-N-butyl phosphate (TNBP) as disclosed in U.S. Pat. No. 4,764,369, but the present invention is not limited thereto.

Preferably, the process is carried out using a solvent/detergent solution comprising TNBP (tri-N-butyl phosphate) and a nonionic detergent, and a virus-inactivating agent particularly preferred for carrying out the present invention is a mixture of TNBP and at least one selected from among polysorbate 80 (Tween 80), polysorbate 20 (Tween 20), Triton X-100, and Triton X-45, but is not limited thereto.

In the present invention, the anion exchange chromatography may be performed under conditions of a pH of 8.0 to 8.25, a flow rate of 129 to 150 cm/hr, a wash volume of 10 CV (column volume) or more, and an elution volume of 2 CV. Here, the fraction that was attached to the column and then separated by the elution buffer may be obtained at 1.5 to 2.0 CV.

Examples of the anion exchange resin used in the anion exchange chromatography step may comprise those substituted with diethylaminoethyl (DEAE), trimethylaminoethyl (TAME), triethylaminoethyl (TEAE), aminoethyl (AE), diethylaminopropyl (ANX), or quaternary ammonium (Q) groups, but are not limited thereto. The anion exchange resin is preferably any one selected from among anion exchange resins having a strongly basic quaternary ammonium group or a weakly basic diethylaminoethyl (DEAE) group, more preferably any one selected from among anion exchange resins having a weakly basic diethylaminoethyl (DEAE) group, most preferably DEAE-650M, but is not limited thereto.

The appropriate volume of the resin used for anion exchange chromatography is controlled by the column dimensions, namely the diameter of the column and the height of the resin, and depends, for example, on the amount of immunoglobulin solution in the solution applied and the binding performance of the resin used. Before performing ion exchange chromatography, the ion exchange resin is preferably equilibrated with a buffer to allow the resin to bind to the counterion thereof.

In the present invention, the anion exchange resin may be exemplified by DEAE-Sepharose gel, and examples of the column buffer may comprise an equilibration buffer, a wash buffer, and an elution buffer as known in the art, such as a sodium phosphate buffer, citrate buffer, acetate buffer, Tris buffer, etc.

In the present invention, the purification method may further comprise (f) performing filtration after step (e), and the filtration is preferably nanofiltration.

The nanofiltration may be performed using a commercially available nanofiltration system, and examples of the filter that may be used preferably comprise SV4 20N manufactured by Pall, Planova 20N manufactured by Asahi Kasei, and the like, but are not limited thereto. This process may be conducted using an appropriate buffer, and is preferably performed using a buffer containing 10 mM Tris and 115 to 130 mM NaCl at a pH of 8.0 to 8.25 under conditions of a temperature of 21±3° C. and a pressure of 0.90±0.08 bar, but the present invention is not limited thereto.

In the present invention, the purification method may further comprise (g) performing dialysis or concentration using an ultrafiltration/diafiltration (UF/DF) system after step (f).

In the present invention, “UF diafiltration” is a technique for removing or obtaining a component (e.g. particles) contained in a target material (solution), and a technique for increasing the purity of a target material using a permeable filter that is separable depending on the molecular weight (molecular size) of the component. Ultrafiltration/diafiltration (UF/DF) may be conducted using a typical UF/DF system, and may be characterized by changing to a constant osmotic pressure, buffer exchange, and concentration control.

A better understanding of the present invention may be obtained through the following examples. These examples are merely set forth to illustrate the present invention, and are not to be construed as limiting the scope of the present invention, as will be apparent to those skilled in the art.

### Example 1: Process for Purification of Factor XIII

### Example 1-1: Preparation of Sample

Imported plasma (NSP) was used for plasma and stored at −70° C. or lower until use. 2000 to 2600 L of plasma was thawed at 17 to 27° C. and then pooled while maintaining the temperature of a plasma collection tank at 1 to 6° C.

Under conditions of 1 to 6° C. and 0.4 to 1.5 kg/cm², centrifugation was performed at 5400 rpm (6281 to 6764 xg) and 390 L/hr when using CF30 and at 6500 rpm (6869 to 7305 xg) and 340 L/hr when using CF12, thereby collecting a cryopaste.

### Example 1-2: Cryopaste Dissolution

6 kg of a dissolution buffer (20 mM sodium citrate+50 mM NaCl, a pH of 7.0) was added to 2 kg of the cryopaste containing FXIII (paste: buffer=1:3), followed by stirring at 25 to 35° C. for 150 to 210 minutes at a stirrer speed adjusted to 250 rpm.

### Example 1-3: First Citrate Precipitation and Dissolution

1 to 3% (w/v) Celpure® 1000 was added to the cryopaste solution containing FXIII at 30° C. dissolved in Example 1-2 and a pH adjuster solution (pH adjuster solution: 0.5 M HCl/0.5 M NaOH) was added thereto to adjust the pH to 6.5 to 7.1. Thereafter, 310 to 350 mM sodium citrate was added thereto, followed by reaction with stirring at 200 to 500 rpm for 20 to 60 minutes.

After completion of reaction, centrifugation was performed at 5000 to 8000 g for 30 minutes while maintaining the temperature at 1 to 10° C., and a precipitate was collected. The precipitate thus collected was added with UPW in an amount corresponding to 5 to 7 times the amount thereof, followed by stirring at 30 to 36° C. for 60 to 120 minutes at a stirrer speed adjusted to 250 rpm.

### Example 1-4: Heat Treatment

3 to 5% (w/v) AlOH3 was added to the solution dissolved in Example 1-3, after which the pH of the processing solution was adjusted to 6.9 to 7.3 using a pH adjuster solution (pH adjuster solution: 0.5 M HCl/0.5 M NaOH).

Thereafter, heat treatment was performed at 50 to 60° C. for 8 to 12 minutes, the temperature was lowered to 25° C. or less, and then the solution was centrifuged (7000 g, 30 minutes, 4° C.). The supernatant was filtered with a glass fiber filter.

### Example 1-5: Second Citrate Precipitation and Dissolution

The temperature of the supernatant obtained in Example 1-4 was adjusted to 27 to 35° C., and the pH thereof was adjusted to 6.5 to 7.1 using a pH adjuster solution (pH adjuster solution: 0.5 M HCl/0.5 M NaOH). Thereafter, 640 to 700 mM sodium citrate was added thereto, followed by reaction with stirring at 200 to 500 rpm for 20 to 100 minutes.

After completion of reaction, centrifugation was performed at 5000 to 8000 g for 30 minutes while maintaining the temperature at 1 to 10° C., and a precipitate was collected.

The precipitate thus collected was added with UPW in an amount corresponding to

20 times the amount thereof (paste: buffer=1:20), followed by stirring at 21° C. for 50 minutes at a stirrer speed adjusted to 250 rpm.

### Example 1-6: Solvent/Detergent (SID) Treatment

In order to inactivate latent virus in the cryopaste solution containing FXIII, solvent/detergent treatment was performed.

Specifically, an SID stock was added so as to achieve 0.3% TNBP and 1% (w/v) polysorbate in the second precipitate solution obtained in Example 1-5, followed by stirring at 21° C. for 270 minutes at 200 rpm.

### Example 1-7: Anion Exchange Chromatography

In order to finally remove impurities from the SID-treated solution obtained in Example 1-6, anion exchange chromatography was performed using a purification column packed with DEAE-650M (Toyopearl) resin.

The column was equilibrated with a 10 mM Tris (pH 8.0 to 8.25) equilibration buffer, and the SID-treated solution was filtered with a 0.22 μm filter (Saitobran P) and loaded onto the column at a linear flow rate of 129 to 150 cm/hr. After completion of loading, washing was performed with a wash buffer (10 mM Tris, 50 to 60 mM NaCl, a pH of 8.0 to 8.25).

After completion of washing, a tai get protein was eluted using an elution buffer (10) mM Tris, 115 to 130 mM NaCl, a pH of 8.0 to 8.25) with sodium chloride added thereto.

### Example 1-8: Nanofiltration

Nanofiltration was performed to remove latent virus from the cryopaste solution containing FXIII. After wetting the nanofilter with water for injection, an integrity test was performed on the filter. The nanofilter subjected to the integrity test was equilibrated while allowing 30 mL or more of an AEX elution buffer (10 mM Tris, 115 to 130 mM NaCl, a pH of 8.0 to 8.25) to flow thereto.

After completion of equilibration, the AEX eluate obtained in Example 1-7 was filtered at a pressure of about 0.90±0.08 bar to afford an ultrafine filtrate. After completion of filtration, washing was performed with a formulation buffer, and the wash and the nanofilter filtrate were collected.

### Example 1-9: Ultrafiltration/Diafiltration (UFIDF)

An ultrafiltration system (Tangential Flow Filtration Membrane System) was used to adjust the protein concentration of the solution collected in Example 1-8 and to exchange the purified protein with a formulation buffer. The membrane (Cut off: 50 kDa, Merck) in the ultrafiltration system was equilibrated using an AEX elution buffer (10 mM Tris, 115 to 130 mM NaCl, a pH of 8.0 to 8.25), after which the target protein was primarily concentrated at TMP of 1.0 bar.

After concentration, a diafiltration buffer (20 mM histidine, 50 mM NaCl, 0.625% arginine-HCl, a pH of 7.3) in a volume cmresponding to 5 to 8 times the volume of the concentrate was continuously exchanged, and as such, the process was stopped when the conductivity and pH fell within reference ranges (reference conductivity: 6.20 to 7.00 mS/cm, reference pH: 7.20 to 7.40). Concentration was performed so that the FXIII titer of the buffer-exchanged sample was 160 IU/mL or more.

### Example 1-10: Formulation and Lyophilization

Polysorbate 20, sucrose, and NaCl were added to the concentrate obtained in Example 1-9, so that the final composition was composed of 20 mM L-histidine, 0.01% polysorbate 20, 70 mM sodium chloride, 3% sucrose, and 0.5% Arg-HCl (pH 7.0), followed by lyophilization.

### Example 2: Confirmation of Optimal Additive and Content in First Citrate Precipitation Step

Based on the results of experiments at set points of predetermined process parameters through design of experiments (DOE), the first precipitate was confirmed to adhere to the impeller, which is determined to cause a problem upon application to a production scale later. Thus, the present inventors tried to find conditions capable of preventing the first precipitate from adhering to the impeller by adding an auxiliary material.

The present inventors ascertained that protein aggregation was reduced in the heat treatment process when using a solid powder, and tried to solve the phenomenon by which the precipitate adheres to the impeller by adding the solid powder. Celpure® 1000, Celpure® 300, and Vitacel® were used as the solid powder (hereinafter,® omitted). Celpure® 1000 and Celpure® 300 were obtained from Sigma-Aldrich (US) (Product Nos. 525227 and 525243), and Vitacel® (composition: 74% of cellulose, 26% of hemicellulose, ::s; 0.5% of lignin) was obtained from JRS (Germany).

The pH of the heat-treated solution was adjusted to 6.6, after which 1, 2, or 3% of each Celpure was added to the heat-treated solution (mL)+sodium citrate (mL), and the first precipitation process was performed a total of 10 runs (Table 3).

TABLE 3
Study plan for selection of solid powder

| | Process parameter | | | | |
|---|---|---|---|---|---|
| | | | Sodium | | |
| | Sodium | | citrate & | | |
| | citrate | Temper- | Cryopaste | Solid powder | |
| Study | Concentration | ature | solution | | Content |
| No. | (mM) | (° C.) | pH | Name | (%) |
| 1 | 330 | 30 | 6.6 | Celpure 1000 | 1 |
| 2 | 330 | 30 | 6.6 | Celpure 300 | |
| 3 | 330 | 30 | 6.6 | Vitacel | |
| 4 | 330 | 30 | 6.6 | Celpure 1000 | 2 |
| 5 | 330 | 30 | 6.6 | Celpure 300 | |
| 6 | 330 | 30 | 6.6 | Vitacel | |
| 7 | 330 | 30 | 6.6 | Celpure 1000 | 3 |
| 8 | 330 | 30 | 6.6 | Celpure 300 | |
| 9 | 330 | 30 | 6.6 | Vitacel | |

| | | | | |
|---|---|---|---|---|
| 10 | 330 | 30 | 6.6 | N/A |

After completion of the first precipitation process, the solid powder, by which the precipitate did not adhere to the impeller, was selected by measuring the properties thereof, and the FXIII titer and total protein content of the selected solid powder were analyzed. Whether the QA/PA result values satisfied the acceptance criteria or whether there was a significant difference between experimental groups was determined.

Based on the results of 10 runs, for Celpure 1000 and Vitacel, the precipitate did not adhere to the impeller from 2%, but for Celpure 300, there was no adhesion phenomenon from 3%.

Therefore, the FXIII titer and total protein content were analyzed for the first precipitate to which Celpure 1000 (2%), Vitacel (2%), and Celpure 300 (3%), prevented the precipitate from adhering to the impeller, were added, and the results thereof are shown in Table 4 below.

TABLE 4
Study results of selected solid powder

| | | QA/PA | | |
|---|---|---|---|---|
| | | Factor XIII | Protein | Specific |
| Study | | activity/CPS | content/CPS | activity |
| No. | Solid powder | 1 mL(IU/mL) | 1 mL(mg/mL) | (IU/mg) |
| 1 | Celpure 1000 (2%) | 1.958 | 21.43 | 0.091 |
| 2 | Vitacel (2%) | 1.749 | 22.28 | 0.079 |
| 3 | Celpure 300 (3%) | 1.979 | 20.59 | 0.096 |
| 4 | N/A | 1.574 | 19.74 | 0.080 |

| | | | |
|---|---|---|---|
| Acceptance criteria | ≥1.409 | ≤25.49 | N/A |

In order to simultaneously compare changes in FXIII titer and total protein content, specific activity values were compared, and the specific activity values showed no difference or a tendency to increase compared to the existing conditions in which solid powder was not added.

Thereamong, Celpure 300 (3%) and Celpure 1000 (2%), which have higher specific activity values than conventional cases, were determined to be suitable. For Celpure 300 (3%), there was no adhesion phenomenon to the impeller, but precipitates were observed to agglomerate into small balls, based on which Celpure 1000 (2%) was ultimately judged to be appropriate.

### INDUSTRIAL APPLICABILITY

According to the present invention, factor XIII can be purified at high yield from a plasma sample or the like through a simple process, and factor XIII can be purified with a high purity of about 80% or more compared to conventional purification methods showing a purity of about 50%. Moreover, since the method of the present invention is applicable to large-scale industrial production, it is regarded as economical and efficient, and also, this method can be useful in the field of prevention and treatment of diseases associated with factor XIII deficiency.

Although specific embodiments of the present invention have been disclosed in detail above, it will be obvious to those skilled in the art that the description is merely of preferable exemplary embodiments and is not to be construed as limiting the scope of the present invention. Therefore, the substantial scope of the present invention will be defined by the appended claims and equivalents thereto.

1. A method of purifying factor XIII (FXIII), comprising:
performing a first salt precipitation by adding diatomite and a salt to a separated plasma sample comprising factor XIII, and then obtaining a precipitate by removing a supernatant;
dissolving the precipitate in a dissolution buffer, and then treating with heat to form a solution; and
performing a second salt precipitation by adding a salt to the solution, and then obtaining a precipitate by removing a supernatant.

2. The method according to claim 1, wherein an amount of the diatomite added in the first salt precipitation is from 0.5 to 5% (w/v) of a total volume.

3. The method according to claim 1, wherein the salt in each of the first salt precipitation and the second salt precipitation is citrate.

4. The method according to claim 3, wherein the citrate is sodium citrate.

5. The method according to claim 4, wherein a concentration of sodium citrate in the first salt precipitation is from 250 to 400 mM, and a concentration of sodium citrate in the second salt precipitation is from 500 to 800 mM.

6. The method according to claim 1, wherein a temperature of the treating with heat is from 40 to 70° C.

7. The method according to claim 1, further comprising, after the second salt precipitation:
treating with a solvent/detergent; and/or
performing anion exchange chromatography.

8. The method according to claim 7, further comprising performing filtration.

9. The method according to claim 8, further comprising performing dialysis or concentration using an ultrafiltration/diafiltration (UF/DF) system, after performing the filtration.
